(19) United States
(12) Patent Application Publication
(10) Pub. No.: US 2012/0309676 A1
(43) Pub. Date: Dec. 6, 2012

(54) Lantibiotic Salts

(76) Inventors: Sjoerd Nicolaas Wadman, Welwyn Garden City (GB); Antony Nicholas Appleyard, Welwyn Garden City (GB)

(21) Appl. No.: 13/576,892

(22) PCT Filed: Feb. 1, 2011
(86) PCT No.: PCT/GB2011/000133

§ 371 Date: Aug. 2, 2012

(30) Foreign Application Priority Data
Feb. 2, 2010 (GB) 2010/15215
Feb. 2, 2010 (GB) PCT/GB2010/000188
Feb. 2, 2010 (TW) 099103071
Aug. 11, 2010 (GB) 1013511.9

(51) Int. Cl. A61K 38/12; A61P 31/04; C07K 7/64; A61P 31/00

(52) U.S. Cl. 514/2.3; 514/2.4; 514/2.6; 514/2.7; 514/21.1; 530/317

(57) ABSTRACT

Described are certain salts of certain lantibiotic compounds, pharmaceutical compositions comprising the same and use of the salts and compositions for the treatment of microbial infection, particularly Methicillin-resistant Staphylococcus aureus (MRSA) infection. The salts have an aqueous solubility of 2.5 mg/mL or more.

This application is related to PCT patent application PCT/GB2010/000188 filed 2 Feb. 2010, TW patent application 099103071 filed 2 Feb. 2010, GF patent application 2010/15215 filed 2 Feb. 2010 and GB patent application GB 1013511.9 filed 11 Aug. 2010; the contents of both of which are incorporated herein by reference in their entirety.

The present disclosure relates to certain novel salts of certain lantibiotic compounds, pharmaceutical compositions comprising same and use of the salts and compositions for the treatment of microbial infection, particularly Methicillin-resistant Staphylococcus aureus (MRSA) infection.

Many antibiotic compounds have been identified from natural sources including microorganisms. Often the antibiotic compounds have a complicated chemical structure and in particular a complicated stereochemical structure.

Actagardine is a natural product prepared from Actinoplanes garbadinensis, and has antibiotic properties, see for example EP0195359, in particular against Streptococcus pyogenes, which causes scarlet fever and strep throat infection. Despite the need for new antibiotics in the 22 years since publication of EP0195359, no antibiotics derived from actagardine have been licensed and marketed.

A new family of compounds based on deoxyactagardine B was recently disclosed in WO 2007/083112. Deoxyactagardine B is prepared from A. liguriae and has a number of distinguishing features from actagardine, in particular the compounds have differences in the amino acid sequence of the core structure. Additionally actagardine contains an oxidised lanthionine bridge in contrast to deoxyactagardine B, wherein all the lanthionine bridges are present in a reduced form. Obviously different genes and biological machinery is required to make the different compounds. Furthermore, these compounds show different activity when tested against a range of common pathogens. In some instances actagardine and certain compounds derived therefrom exhibit greater activity against a given pathogen than deoxyactagardine B and derivatives thereof. Interestingly, against certain other pathogens deoxyactagardine B and compounds derived therefrom exhibit greater activity than actagardine and derivatives thereof.

Actagardine activity against MRSA when measured by a standard test, such as minimum inhibitory concentrations (MICs), may be as high as about 32 μg/mL, depending on the strain tested. Thus actagardine has only low to moderate activity against MRSA because the higher the MIC value the less antimicrobial activity the compound has.

Deoxyactgardine B activity against MRSA when measured by a standard test, such as minimum inhibitory concentrations (MICs), may have an activity as high as about 32 μg/mL, depending on the strain tested. Thus deoxyactagardine B has only low to moderate activity against MRSA.

MRSA is a bacterium responsible for difficult-to-treat infections in humans and animals. The particular strain(s) of S. aureus that is labelled MRSA is/are resistant to a large group of antibiotics called beta-lactams, which include the penicillins and cephalosporins.

The strain(s) received a significant amount of attention in the media and was branded a “superbug”. Patients with open wounds, those who have procedures involving invasive devices, and those with a weakened immune system are most at risk of infection, especially during hospitalization. The infection is highly contagious and if it is identified on a hospital ward the ward may be closed until it is decontaminated.

Thus antimicrobial compounds with activity against MRSA would be particularly useful.

Deoxyactagardine B (3,5-dichlorobenzylamine) monocarboxamide and actagardine (3,5-dichlorobenzylamine) monocarboxamide have activity against a range of gram positive bacteria, including Methicillin sensitive S. aureus. and for example have activity against one or more strains of MRSA of 8 μg/mL or less, such as 4 or 2 μg/mL, which represents at least a 2 fold, such as a 4 or 8 fold, increase in activity over the parent actagardine or deoxyactagardine B compounds. In addition, the fact that they have activity against a range of gram positive bacteria makes them therapeutically useful.

However, the aqueous solubility of these compounds is low, for example about 0.25 mg/mL after lyophillisation, which thereby renders the compounds unsuitable for formulation by parenteral routes.

At moderate pH values, for example between pH 4 and pH 8, the compound exists as a zwitterion. A strong acid, such as hydrochloric acid, would generally break the zwitterion in the compound and form a salt which would normally be expected to exhibit enhanced aqueous solubility. Surprisingly salt formation with strong acids such as hydrochloric acid and phosphoric acid did not give material with substantially increased aqueous solubility. Generally a solubility of at least 2 mg/mL and probably 5 mg/mL is required for the preparation of parenteral formulations

The present inventors have found a small number of salts with good aqueous solubility, thereby allowing them to be formulated for parenteral administration.

Thus there is provided a salt of a compound of formula (I):

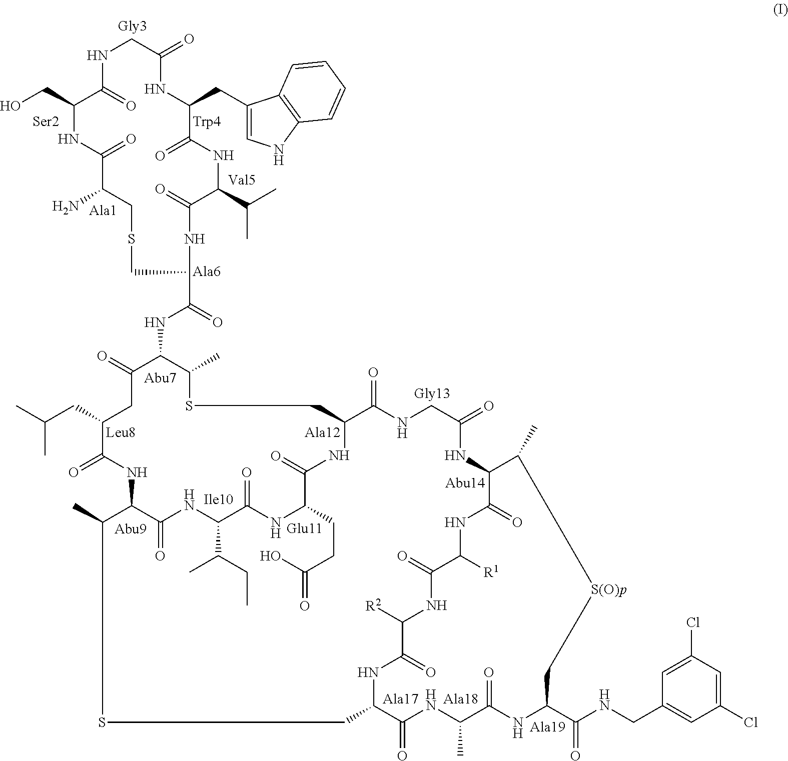

wherein
R¹ together with the carbon to which it is attached and the alpha-nitrogen and alpha-carbonyl represents an amino acid residue;
R² together with the carbon to which it is attached and the alpha-nitrogen and alpha-carbonyl represents an amino acid residue; and
p represents 0 or 1,
   wherein the salt is formed from a small organic amine comprising at least one hydroxyl group.

In order to be useful for the preparation of parenteral formulations a compound, or salt of a compound generally requires a solubility higher than 2.5 mg/mL, such as 5 mg/ml.

Advantageously, salts of the present disclosure have an aqueous solubility of 2.5 mg/mL or more, such as in the range 2.5 to 50 mg/mL, in particular a solubility in the range 2.5 to 20, more particularly in the range 5 to 15, such as 10, which represents at least a 20-fold increase in aqueous solubility over the parent compound.

Solubility as employed herein is used generically to refer to a distribution of a compound of formula (I) in a solvent (for example water, saline or an aqueous isotonic solution such as glucose) such that the formulation/composition resulting therefrom is substantially free of visible aggregates and for example is substantially transparent when viewed by the naked eye. Such a formulation would therefore include a whole range of formats which are suitable for infusion and/or injection such as solutions, suspensions, colloids and the like.

Regardless of how the salts of the present invention are formulated, increased solubility (ability to be distributed in a solvent such as aqueous environment) is an important property.

Advantageously, salts of the present disclosure tend to have higher solubility than inorganic salts.

Table 1 shows the solubility of certain salts according to the present disclosure

TABLE 1
Summary of salts found exhibiting aqueous solubility >1 mg/mL

| Details | Synthesis | |
|---|---|---|
| Salt form | Solubility (mg/mL) | pH of saturated solution |
| N-ethyl-D-glucamine salt | 11.16* | 8.43 |
| ethanolamine salt | 10.5* | 8.74 |
| diethanolamine salt | 6.94* | 8.78 |
| N-methyl-D-glucamine | 6.87* | 8.43 |
| salt | | |
| triethanolamine salt | 1.04 | 8.43 |
| tromethamine salt | 2.38 | 8.53 |

*calculations made from two determinations

Other properties of certain salts according to the disclosure include the ability to make clear “solutions” (see table 3 in the experimental section) and/or the ability to control the final pH of the “solution” formed.

FIG. 6 shows the optimum range of pH values to obtain the best solubility (mg/ml). The ranges shown are between 6.5-9 such as 7-8.5. Nevertheless solubility is not the only property that is important when selecting a drug candidate.

### BRIEF DESCRIPTION OF THE FIGURES

FIG. 1 is a graph plot representing the in vivo efficacy of the compound Example 1 in a mouse bacteraemia model over 7 days

FIG. 2 is a graph plot evaluating the in vivo efficacy of the compound Example 1 in the treatment of bacterial tissue infections using a neutropaenic mouse thigh model

FIG. 3 is a graph plot representing dose dependency in the compound Example 1 and vancomycin

FIG. 4 is a graph plot depicting the in vivo plasma half-life of the compound Example 1 in mice

FIG. 5 is an HPLC chromatogram for stability in aqueous solution of the compound Example 1

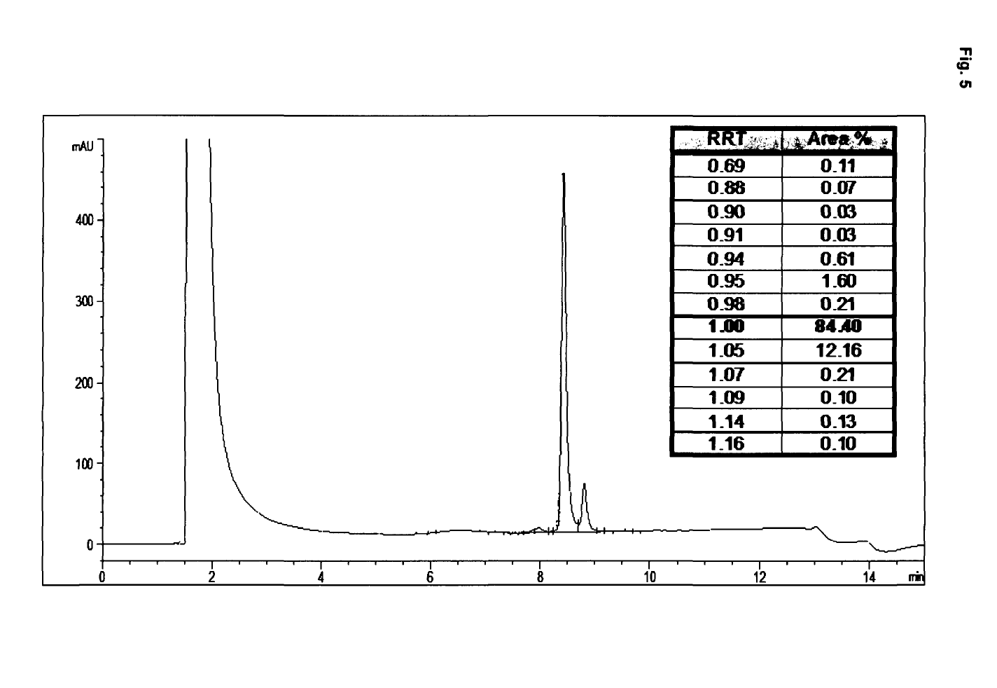

FIG. 6 is a plot graph of solubility vs. pH for the compound Example 1

### DETAILED DESCRIPTION

In one embodiment p is 1.

In one embodiment p is 0.

In one embodiment R¹ together with the carbon to which it is attached and the alpha-nitrogen and alpha carbon is a proteinogenic amino acid.

In one embodiment R¹ together with the carbon to which it is attached and the alpha-nitrogen and alpha-carbonyl represents leucine or valine.

In one embodiment R² together with the carbon to which it is attached and the alpha-nitrogen and alpha carbon is a proteinogenic amino acid.

In one embodiment R² together with the carbon to which it is attached and the alpha-nitrogen and alpha-carbonyl represents isoleucine or valine.

In one embodiment R¹ together with the carbon to which it is attached and the alpha-nitrogen and alpha-carbonyl represents valine and R² together with the carbon to which it is attached and the alpha-nitrogen and alpha-carbonyl represents isoleucine.

In one embodiment R¹ together with the carbon to which it is attached and the alpha-nitrogen and alpha-carbonyl represents leucine and R² together with the carbon to which it is attached and the alpha-nitrogen and alpha-carbonyl represents valine.

In one embodiment R¹ together with the carbon to which it is attached and the alpha-nitrogen and alpha-carbonyl represents valine and R² together with the carbon to which it is attached and the alpha-nitrogen and alpha-carbonyl represents valine.

In one embodiment R¹ together with the carbon to which it is attached and the alpha-nitrogen and alpha-carbonyl represents leucine and R² together with the carbon to which it is attached and the alpha-nitrogen and alpha-carbonyl represents isoleucine.

In one embodiment the compound is:

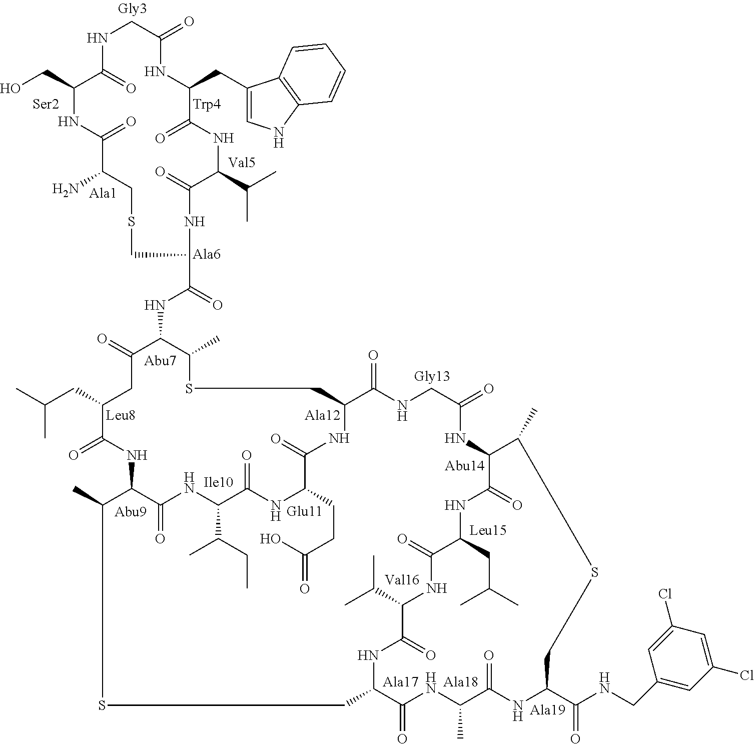

In one embodiment, the compound is not deoxyactagardine B (3,5-dichlorobenzyl amine) monocarboxamide N-methyl-D-glucamine salt.

The formation of salts of lantibiotic type B compounds with good solubility is somewhat unpredictable. Thus it is surprising that salts of the present invention generally have an aqueous solubility of at least 10 mg/mL for example 20 mg/mL, such as 30-40 mg/mL, in particular 50 mg/mL.

Small organic amine as employed herein is intended to refer to a carbon containing compound containing not more than 10 carbon atoms, for example 9, 8, 7, 6 or 5 carbon atoms, wherein the entity contains an amine somewhere therein.

In one embodiment the organic amine compound with which the salt is formed comprises no more than 8, in particular 6, 7 or 8 carbon atoms. In one embodiment the organic amine comprises no more than 4, 5 or 6 carbon atoms.

In one embodiment the organic amine comprises one, two, three, four or five hydroxyl groups. In one embodiment the organic amine comprises one, two, three or four hydroxyl groups, in particular three or four hydroxyl groups.

A small organic amine comprising at least one hydroxyl group as employed herein may refer to the material from which the salt is prepared.

In one embodiment, the amine of the organic amine compound is a group —NH₂. In one embodiment, the amine of the organic amine compound is a group —NHR¹, where —R¹ is an alkyl group having at least 2 carbon atoms.

In one embodiment a hydroxyl in the organic amine is bonded to a carbonyl carbon therein to form a carboxylic acid, at least in the starting material from which the salt is formed. Thus in one embodiment the organic amine entity comprises a carboxylic acid. In one embodiment the organic amine entity comprises one carboxylic acid.

In one embodiment the salt is formed from an amino alcohol.

In one embodiment the hydroxyl group is attached to a carbon situated beta relative to the nitrogen of the amine.

In one embodiment the organic amine is selected from glucamine, such as N-methyl glucamine or N-ethyl glucamine, ethanolamine, diethanolamine and arginine. In one embodiment the organic amine is selected from N-ethyl glucamine, diethanolamine and arginine. Additionally or alternatively, the organic may be N-methyl-L-glucamine.

Thus, in one embodiment there is provided a salt of a compound disclosed herein wherein the organic amine is a glucamine, for example N-methyl glucamine or N-ethyl glucamine, such as N-methyl glucamine.

In one embodiment the organic amine is N-ethyl glucamine.

In one embodiment the organic amino hydroxyl compound is a glucosamine.

In another embodiment there is provided a salt of a compound disclosed herein wherein the organic amine is ethanolamine.

In one embodiment the salt is not triethanolamine.

In a further embodiment there is provided a salt of a compound disclosed herein wherein the organic amine is selected from arginine, lysine or 2-amino-1,3-propanediol.

In one embodiment the organic amine is not ethanolamine. In one embodiment the organic amine is not N-methyl-D-glucamine. In one embodiment the organic amine is not glutamate.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. The salts of the disclosure may form solvates (e.g. hydrates) and the disclosure also includes all such solvates.

In one embodiment there is provided a solvate, such as a hydrate, of a salt of a compound disclosed herein.

With regard to stereoisomers, the salts of the present disclosure have more than one asymmetric carbon atom. In the general formula (I) as drawn, the solid wedge shaped bond indicates that the bond is above the plane of the paper. The broken bond indicates that the bond is below the plane of the paper.

The salts of the compound of formula (I) may be in crystalline or amorphous form. Furthermore, some of the crystalline forms of the salts may exist as polymorphs, all forms which are included in the present disclosure. Having said this, generally the compounds are amorphous.

The salts of the present invention are prepared by reacting the compound described herein with a base in an appropriate solvent.

Typically, a pharmaceutically acceptable salt may be readily prepared by using a desired base, as appropriate. The salt may precipitate from solution and be collected by filtration or may be recovered by evaporation of the solvent, for example, a compound of formula (I) may be dissolved in a suitable solvent, for example an alcohol such as methanol, or t-butanol and the base may be added in the same solvent or another suitable solvent. The resulting salt may then be precipitated directly, or by addition of a less polar solvent such as diisopropyl ether or hexane, and isolated by filtration.

The salt can be prepared by dissolving a compound disclosed in the present specification in a suitable alcohol solvent, such t-butanol in the presence of one, two or three equivalents of a small organic amine comprising at least one hydroxyl group.

Thus there is provided a process of producing a salt in solution comprising dissolving the parent compound in a suitable organic polar solvent in the presence of an appropriate amount of salt-forming partner.

In one embodiment the solvent is t-butanol and/or DMSO.

In one embodiment the solvent is t-butanol.

In one embodiment the solvent is t-butanol and/or water for example 20% w/w or less water, such as 10% w/w or less, in particular 5% w/w or less water.

In one embodiment the salt-forming partner is provided as a greater than 1 equivalent such as in the range 0.5 to 3.5 for example 1 to 3 equivalents, such as 1, 1.5, 2 or 2.5 equivalents, in particular 1.1, 1.2, 1.3, 1.4, 1.5, etc.

The compounds described herein are monobasic and thus when more than one equivalent of the organic amine is employed the resultant entity may in fact comprise a true salt and the additional amine in admixture. Nevertheless the present invention extends to such compositions (or admixtures), which may be beneficial for improving the solubility.

In one embodiment the salt of the present disclosure is provided by evaporating the solvent, for example under reduced pressure, for example employing a rotary evaporator.

Lyophilisation (freeze-drying) can be employed in preparation of the salt to provide a lyophilised product of the present disclosure.

Thus there is provided a process of preparing a salt of a compound disclosed herein comprising the step of dissolving the parent compound and a salt-forming partner in a suitable solvent, and lyophilising to provide a lyophilised product of the present disclosure.

Thus in one embodiment there is provide a composition, for example a lyophillised product comprising a salt of a compound of formula (I) and a small organic amine and an excess of said amine.

In one embodiment the salt or lyophilised product of the present disclosure comprises less than 10% w/w, for example 5% w/w or less, such as 3% w/w or less water.

In one embodiment the salt or lyophilised product of the present disclosure comprises less than 5% w/w, for example 2% w/w or less, such as 1% w/w or less, solvent, such as t-butanol.

The lyophilisation product of the present disclosure may comprise small amounts of pH adjustment agents, for example HCl or TRIS buffer.

Alternatively spray-drying can be employed to extract the salt from solution. In one embodiment the salt of the present disclosure is spray-dried, for example to provide a material with suitable flow properties.

In one aspect the salt disclosed herein is spray dried with one or more excipients to provide particles that are agglomerations of the salt and the excipients.

The salts of the disclosure may be formulated for administration in any convenient way for use in human or veterinary medicine and the disclosure therefore includes within its scope pharmaceutical compositions comprising a salt of the disclosure for use in human or veterinary medicine. Such compositions may be presented for use in a conventional manner with the aid of one or more suitable excipients, diluents and/or carriers. Acceptable excipients, diluents and carriers for therapeutic use are well known in the pharmaceutical art, and are described, for example, in Remington's Pharmaceutical Sciences, Mack Publishing Co. (A. R. Gennaro edit. 1985). The choice of pharmaceutical excipient, diluent and/or carrier can be selected with regard to the intended route of administration and standard pharmaceutical practice. The pharmaceutical compositions may comprise as (or in addition to) the excipient, diluent and/or carrier any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

For some embodiments, the salts of the present disclosure may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO 91/11172, WO 94/02518 and WO 98/55148.

In one embodiment the formulation comprises up to 15% w/w of a cyclodextrin.

In one aspect, the invention provides a pharmaceutical composition comprising a therapeutically effective amount of a salt of the present disclosure and a pharmaceutically acceptable excipient, diluent and/or carrier (including combinations thereof). The salts of the disclosure may be milled using known milling procedures such as wet milling to obtain a particle size appropriate for tablet formation and for other formulation types. Finely divided (nanoparticulate) preparations of the salts of the invention may be prepared by processes known in the art, for example see International Patent Application No. WO 02/00196 (SmithKline Beecham).

The routes for administration (delivery) include, but are not limited to, one or more of: oral (e.g. as a dry powder/free flowing particulate formulation, tablet, capsule, or as an ingestable solution or suspension) rectal, buccal, and sublingual. The compositions of the disclosure include those in a form especially formulated for parenteral, oral, buccal, rectal, topical, implant, ophthalmic, nasal or genito-urinary use. In one aspect of the invention, the agents are delivered orally, hence, the agent is in a form that is suitable for oral delivery.

In some instances it may be possible to deliver the salts of the disclosure by a topical, parenteral (e.g. by an injectable form) or transdermal route, including mucosal (e.g. as a nasal spray or aerosol for inhalation), nasal, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral).

There may be different composition/formulation requirements depending on the different delivery systems or different routes of administration. By way of example, the pharmaceutical composition of the present disclosure may be formulated to be delivered using a mini-pump or by a mucosal route, for example, as a nasal spray or aerosol for inhalation or ingestable solution, or parenterally in which the composition is formulated in an injectable form, for delivery by, for example, an intravenous, intramuscular or subcutaneous route. Alternatively, the formulation may be designed to be delivered by both routes. Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously.

For parenteral administration, the compositions may be best used in the form of a sterile aqueous solution which may contain other substances, for example enough salts or saccharides, in particular a monosaccharide, to make the solution isotonic with blood. Examples of parenteral administration include one or more of: intravenously, intraarterially, intraperitoneally, intrathecally, intraventricularly, intraurethrally, intrasternally, intracranially, intramuscularly or subcutaneously administering the agent, and/or by using infusion techniques.

In one embodiment the formulation is adapted for delivery by infusion or slow injection.

In one embodiment the formulation is adapted for delivery by bolus injection.

In one embodiment the formulation to be administered is a clear solution.

For buccal or sublingual administration the compositions may be administered in the form of tablets or lozenges which can be formulated in a conventional manner.

The salts of the disclosure can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

The salts of the disclosure may also be presented for human or veterinary use in a form suitable for oral or buccal administration, for example in the form of solutions, gels, syrups, mouth washes or suspensions, or a dry powder for constitution with water or other suitable vehicle before use, optionally with flavouring and colouring agents.

Solid compositions such as tablets, capsules, lozenges, pastilles, pills, powder, pastes, granules, bullets or premix preparations may also be used. Solid and liquid compositions for oral use may be prepared according to methods well known in the art. Such compositions may also contain one or more pharmaceutically acceptable carriers and excipients which may be in solid or liquid form.

The tablets may contain excipients such as microcrystalline cellulose, lactose, sodium citrate, calcium carbonate, calcium sulphate, dibasic calcium phosphate and glycine, mannitol, pregelatinised starch, corn starch, potato starch, disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Solid compositions of a similar type may also be administered in gelatin or HPMC (hydroxypropyl methylcellulose) capsules. Suitable excipients in this regard include microcrystalline cellulose, lactose, calcium carbonate, calcium sulphate, dibasic calcium phosphate and, mannitol, pregelatinised starch, corn starch, potato starch or high molecular weight polyethylene glycols.

For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

Capsules, may be filled with a powder (of medicament alone or as blend with selected filler(s)) or alternatively a liquid, each comprising one or more salts of the present disclosure and optionally a carrier. Where the capsule is filled with a powder the salts of the present disclosure and/or the carrier may be milled or micronised to provide material with an appropriate particle size.

Salts of the disclosure may be coated, for example with as an enteric coating when administered orally as a tablet or capsule. The tablet or capsule, as appropriate, may, for example be coated by a thin film such as a EUDRAGIT® film available from Rohm Pharma Polymers, which allows controlled dissolution in the gastrointestinal tract. The films are available as cationic polymers such as EUDRAGIT® E 100 (aminoalkyl methacylate copolymers) or as anionic acrylic polymers such as EUDRAGIT® L (methacrylic acid copolymers) and EUDRAGIT S.

Permeable acrylic polymers such as EUDRAGIT® RL (amino methacrylate copolymer) and EUDRAGIT® RS are also available.

These coating formulations may be prepared as an aqueous dispersion including optional ingredients such as talc, silicone antifoam emulsion, polyethylene glycol. Alternatively the coating formulation may be prepared as an organic polymer solution.

Alternatively, tablets may be coated using OPADRY® (Surelease®) coating systems, available from Colorcon. Aqueous systems generally comprise up to 15% w/w of OPADRY®. Organic solvent systems generally comprise up to 5% w/w of OPADRY®.

The coatings may be prepared by known techniques, for example by; 1. weighing the required quantity of OPADRY® film coating system, 2. weighing the required quantity of water or other solvent(s) into a mixing vessel, 3. with a mixing propeller in the centre of the vessel and as close to the bottom of the vessel as possible, stirring the solvents to form a vortex without drawing air into the liquid, 4. steadily and quickly adding the OPADRY® powder to the vortex, avoiding powder flotation on the liquid surface, 5. increasing the stirrer speed in order to maintain the vortex, if required, and 6. after all the powder ingredients have been added, reducing the mixer speed and continuing mixing for approximately 45 minutes.

Coatings can be applied by known techniques, using tablet coating machines.

The thickness of the coating applied is generally in the range 5 to 35 microns such as 10 to 30 microns, more specifically 10 or 20 microns, depending on the required effect.

Alternatively, the tablet or a capsule, as appropriate, may be filled into another capsule (preferably a HPMC capsule such as Capsugel®) to provide either a tablet in capsule or capsule in capsule configuration, which when administered to a patient yields controlled dissolution in the gastrointestinal tract thereby providing a similar effect to an enteric coating.

Thus in one aspect the disclosure provides a solid dose formulation of a salt of the present disclosure, for example where the formulation has an enteric coating.

In another aspect the disclosure provides a solid dose formulation comprising a protective capsule as outer layer, for example as a tablet in a capsule or a capsule in a capsule. The enteric coating may provide an improved stability profile over uncoated formulations.

Having said this it is believed that the salts of the present disclosure are not particularly susceptible to degradation by stomach acid or intestinal enzymes in vivo.

The salts of the disclosure may also be administered orally, in veterinary medicine, in the form of a liquid drench such as a solution, suspension or dispersion of the active ingredient together with a pharmaceutically acceptable carrier or excipient.

The salts of the invention may also, for example, be formulated as suppositories e.g. containing conventional suppository bases for use in human or veterinary medicine or as pessaries e.g. containing conventional pessary bases.

In one embodiment the formulation is provided as a formulation for topical administration including inhalation.

Suitable inhalable preparations include inhalable powders, metering aerosols containing propellant gases or inhalable solutions free from propellant gases. Inhalable powders according to the disclosure containing the active substance may consist solely of the abovementioned active substances or of a mixture of the abovementioned active substances with physiologically acceptable excipient.

These inhalable powders may include monosaccharides (e.g. glucose or arabinose), disaccharides (e.g. lactose, saccharose, maltose), oligo- and polysaccharides (e.g. dextranes), polyalcohols (e.g. sorbitol, mannitol, xylitol), salts (e.g. sodium chloride, calcium carbonate) or mixtures of these with one another. Mono- or disaccharides are preferably used, the use of lactose or glucose, particularly but not exclusively in the form of their hydrates.

Particles for deposition in the lung require a particle size less than 10 microns, such as 1-9 microns suitably from 0.1 to 5 μm, particularly preferably from 1 to 5 μm.

The propellant gases which can be used to prepare the inhalable aerosols are known from the prior art. Suitable propellant gases are selected from among hydrocarbons such as n-propane, n-butane or isobutane and halohydrocarbons such as chlorinated and/or fluorinated derivatives of methane, ethane, propane, butane, cyclopropane or cyclobutane. The above-mentioned propellant gases may be used on their own or in mixtures thereof.

Particularly suitable propellant gases are halogenated alkane derivatives selected from among TG11, TG12, TG134a and TG227. Of the abovementioned halogenated hydrocarbons, TG134a (1,1,1,2-tetrafluoroethane) and TG227 (1,1,1,2,3,3,3-heptafluoro propane) and mixtures thereof are suitable for use in formulations of the present invention.

The propellant-gas-containing inhalable aerosols may also contain other ingredients such as co-solvents, stabilisers, surface-active agents (surfactants), antioxidants, lubricants and means for adjusting the pH. All these ingredients are known in the art.

The propellant-gas-containing inhalable aerosols according to the invention may contain up to 5% by weight of active substance. Aerosols according to the disclosure may contain, for example, 0.002 to 5% by weight, 0.01 to 3% by weight, 0.015 to 2% by weight, 0.1 to 2% by weight, 0.5 to 2% by weight or 0.5 to 1% by weight of active.

The salts of the disclosure may also be used in combination with other therapeutic agents. The disclosure thus provides, in a further aspect, a combination comprising a salt of the present disclosure together with a further therapeutic agent. The combination may, for example be a combination of a salt of the compound of formula (I) and an antibiotic, such as vancomycin, a beta-lactam (such as a cephalosporin), an aminoglycoside, a macrolide, a tetracyline, a lipopeptide, an oxazolidinone and/or an anti-inflammatory such as a steriod. The combination may be provided as a co-formulation or simply packaged together as separate formulations, for simultaneous or sequential delivery.

In one embodiment there is provided salts of the present disclosure in combination with a further therapeutic agent.

It is to be understood that not all of the compounds/salts of the combination need be administered by the same route. Thus, if the therapy comprises more than one active component, then those components may be administered by different routes.

The individual components of such combinations may be administered either sequentially or simultaneously in separate or combined pharmaceutical formulations by any convenient route.

When administration is sequential, either the salt of the disclosure or the second therapeutic agent may be administered first. When administration is simultaneous, the combination may be administered either in the same or a different pharmaceutical composition.

The combinations referred to above may conveniently be presented for use in the form of a pharmaceutical formulation and thus pharmaceutical formulations comprising a combination as defined above together with a pharmaceutically acceptable carrier or excipient comprise a further aspect of the disclosure.

When combined in the same formulation it will be appreciated that the two compounds/salts must be stable and compatible with each other and the other components of the formulation. When formulated separately they may be provided in any convenient formulation, in such manner as are known for such compounds in the art.

The compositions may contain from 0.01-99% of the active material. For topical administration, for example, the composition will generally contain from 0.01-10%, more such as 0.01-1% of the active material.

When a salt of the disclosure is used in combination with a second therapeutic agent active against the same disease state the dose of each compound/salt may be the same or differ from that employed when the compound/salt is used alone. Appropriate doses will be readily appreciated by those skilled in the art. It will also be appreciated that the amount of a salt of the disclosure required for use in treatment will vary with the nature of the condition being treated and the age and the condition of the patient and will be ultimately at the discretion of the attendant physician or veterinarian.

Typically, a physician will determine the actual dosage which will be most suitable for an individual subject. The specific dose level and frequency of dosage for any particular individual may be varied and will depend upon a variety of factors including the activity of the specific salt employed, the metabolic stability and length of action of that salt, the age, body weight, general health, sex, diet, mode and time of administration, rate of excretion, drug combination, the severity of the particular condition, and the individual undergoing therapy.

For oral and parenteral administration to humans, the daily dosage level of the agent may be in single or divided doses. For systemic administration the daily dose as employed for adult human treatment it will range from 2-100 mg/Kg body weight, such as 5-60 mg/Kg body weight, which may be administered in 1 to 4 daily doses, for example, depending on the route of administration and the condition of the patient. When the composition comprises dosage units, each unit will preferably contain 100 mg to 1 g of active ingredient. The duration of treatment will be dictated by the rate of response rather than by arbitrary numbers of days.

In one embodiment the treatment regime is continued for 1, 2, 3, 4, 5, 6, 7, 8, 9, 10, 11, 12, 13, 14, 15, 16, 17, 18, 19, 20, 21 or more days.

As described above, the salts of the present disclosure may be employed in the treatment or prophylaxis of humans and/or animals.

In another aspect, the invention provides a pharmaceutical composition comprising, a therapeutically effective amount of a salt of the present disclosure and a pharmaceutically acceptable excipient, diluent and/or carrier for use in therapy, and in particular, in the treatment of human or animal subjects suffering from a condition susceptible to amelioration by an antimicrobial compound.

There is further provided by the present disclosure a process of preparing a pharmaceutical composition, which process comprises mixing a salt of the disclosure or a pharmaceutically acceptable derivative thereof, together with a pharmaceutically acceptable excipient, diluent and/or carrier.

In one embodiment the salts of the present disclosure are useful in treatment, for example in the treatment of gram positive bacterial infections.

In one embodiment the salts of the present disclosure is useful in the treatment of skin infections, in particular bacterial skin and soft tissue infection.

In one aspect, the salts of the present disclosure are suitable for use in therapy, for example, for treatment of microbial infections such as bacteraemia, pneumonia and microbial infection of soft tissue including surgical wounds, in particular Staphylococcal infections including MRSA infection.

In one embodiment salts of the present disclosure are useful for the treatment of Enterococcal infections including E. faecalis and E. faecium infection, for example skin and skin structure infections, endocarditis, urinary tract infection and sepsis.

In one embodiment the salts of the present disclosure are useful for the treatment of S. pyogenes, for example skin infections such as impetigo, erysipelas and cellulitis, throat infections, scarlet fever, and acute glomerulonephritis.

In one embodiment salts of the present disclosure may be useful in the treatment of S. pneumoniae infection, for example pnuemonia, acute sinusitus, otitis media, meningitis, bacteremia, osteomylitis, septic arthritis and endocarditis.

In one aspect salts of the present disclosure may be employed for controlling bacterial overgrowth syndrome. Overgrowth syndrome (BOS) occurs when the normally low bacterial colonization in the upper GI tract and/or lower intestines significantly increases.

In one aspect, the disclosure provides use of salts of the present disclosure in therapy, for example, for treatment of microbial infections such as C. difficile infection, in particular diarrhoea associated therewith, or one or more microbial infections described herein, particularly by oral delivery of a salts of the present disclosure.

In one aspect there is provided use of salts of the present disclosure for the prophylaxis, treatment or maintenance of IBS (irritable bowel syndrome). See for example Rifaximin Treatment for Symptoms of Irritable Bowel Syndrome. Andrea L. Fumi and Katherine Trexler, The Annals of Pharmacotherap, 2008, 4, 408.

In one embodiment the salts of the present disclosure may be useful in the treatment of ulcerative colitis including prophylactic treatment to prevent recurrence thereof. The compounds may be particularly suitable for the treatment of steroid refractory ulcerative colitis. See for example steroid-refractory ulcerative colitis treated with corticosteroids, metronidazole and vancomycin: a case report J. Miner, M. M Gillan, P. Alex, M Centola, BMC Gastroenterology 2005, 5:3.

The salts of the present disclosure may be particularly useful for long term treatment.

In one aspect there is provided a salt of the present disclosure or a composition comprising same for use in treatment or prophylaxis for example the treatment or prophylaxis of any one the indications described herein.

In one aspect there is provided a salt of the present disclosure or a composition comprising the same for the manufacture of a medicament for one or more of the indications defined above.

In one aspect there is provided a method of treatment comprising the step of administering a therapeutically effective amount of a salt of the present disclosure or a pharmaceutical composition containing the same to a patient (human or animal) in need thereof, for example for the treatment of an infection/illness or disease as described herein.

There is provided a pharmaceutical composition comprising a salt of the present disclosure and a pharmaceutically acceptable excipient.

In one aspect there is provided a salt as defined above or a composition comprising the same for use in treatment.

In one aspect there is provided a salt as defined above or a composition comprising the same for use as an antimicrobial agent.

There is also provided a salt or composition as described above for use in the treatment of the microbial infection, such as S. aureus including MRSA, E. faecalis, E. faecium, S. pyogenes, S. pneumoniae and/or C. difficile.

In one aspect there is provided a method of treatment comprising administering a therapeutically effective amount of a salt or composition as defined above to a patient in need thereof.

In one aspect there is provided a method of treatment as described above wherein said method is employed in the treatment of the microbial infection S. aureus infection such as MRSA, E. faecalis, S. pyogenes, S. pneumoniae and/or C. difficile.

In the context of this specification “comprising” is to be interpreted as “including”.

Aspects of the invention comprising certain elements are also intended to extend to alternative embodiments “consisting” or “consisting essentially” of the relevant elements.

Where technically appropriate embodiments may be combined and thus the disclosure extends to all permutations/combinations of the embodiments provided herein.

Preferences given for salts of compounds of formula (I) may equally apply to other salts of the invention, disclosed herein, as technically appropriate.

### EXAMPLES

In compound 1 below the substituent shown is linked to the DAB entity through the C terminus and therefore the specific substituents shown correspond to the C-terminal modification in compounds of formula (I).

### Compound 1: Deoxyactagardine B (3,5-dichlorobenzylamine) monocarboxamide

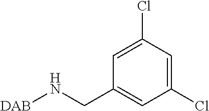

Deoxyactagardine B [DAB] (200 mg), 3,5-dichlorobenzylamine (38 mg) and diisopropylethylamine (35 μL) were dissolved in dry dimethylformamide (1 mL). A solution of benzotriazole-1-yl-oxy-tris-pyrrolidino-phosphonium hexafluorophosphate (PyBOP) (84 mg) in dry DMF (2 mL) was added portionwise. The reaction was followed by analytical hplc (See Table 1) and PyBOP was added until the starting material had been consumed.

TABLE 1
Analytical HPLC conditions for the separation of lantibiotic (e.g.
actagardine, actagardine B, or deoxy-actagardine B) and
diaminoalkane derivatised products.

| Column: | Zorbax 5μ C18(2) 150 × 4.6 mm | | | | |
|---|---|---|---|---|---|
| Mobile Phase A: | 30% Acetonitrile in 20 mM potassium phosphate | | | | |
| | buffer pH 7.0 | | | | |
| Mobile Phase B: | 65% Acetonitrile in 20 mM potassium phosphate | | | | |
| | buffer pH 7.0 | | | | |
| Flow rate: | 1 mL/min | | | | |
| Gradient: | Time 0 min | 100% | A | 0% | B |
| | Time 10 min | 0% | A | 100% | B |
| | Time 11 min | 0% | A | 100% | B |
| | Time 11.2 min | 100% | A | 0% | B |
| | Cycle time 15 min | | | | |

| | |
|---|---|
| Injection volume: | 10 μL |
| Detection: | 210 nm |

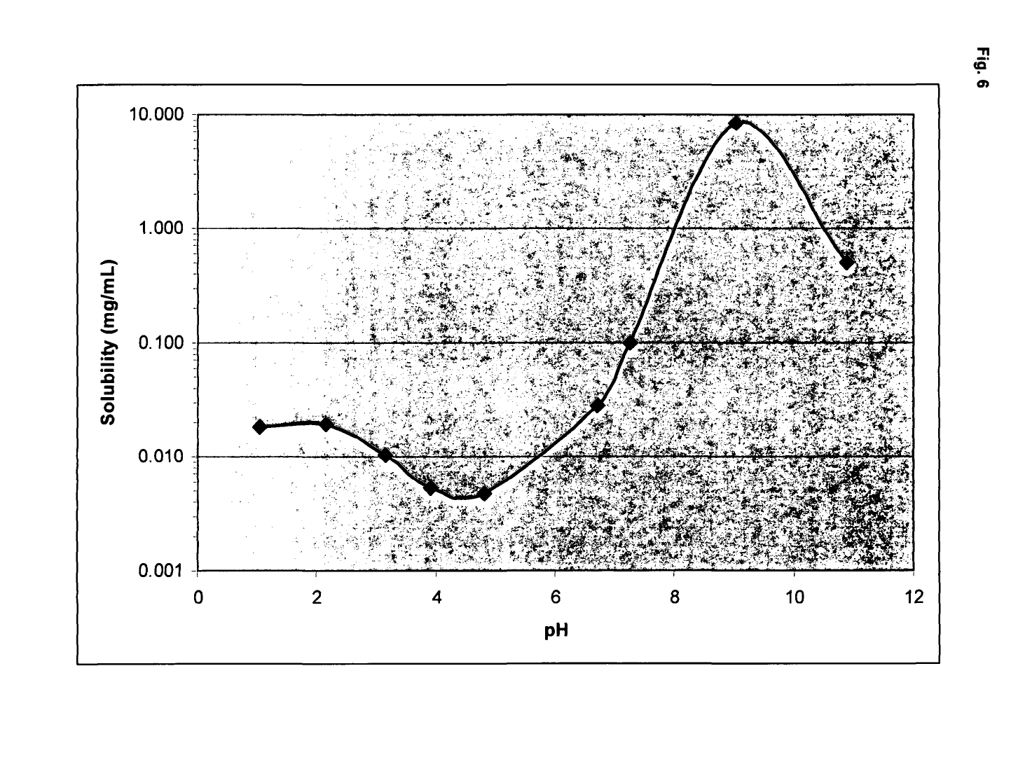

The crude reaction mixture was poured into 30% aqueous methanol and the resulting solution was loaded on to a Varian Bond Elut C18 column (30 g). The column was then washed sequentially with 50%, 60%, 70%, 80%, 90% aqueous methanol, with most of the desired material eluting in the 70% fraction (FIG. 6) Column chromatography on silica gel (eluent dichloromethane:ethanol:ammonia 10:8:1) gave material of >90% purity by U.V. at 210 nm. Yield 107 mg (50%). Mass calculated for (M+2H)⁺² 1015.5. found 1015.57. Calculated for [M+H+Na]² 1026, found 1025.32

Samples were analysed by LC-MS using the conditions described in Table 2.

TABLE 2
LC/MS conditions for the analysis of lantibiotic (e.g. deoxy-actagardine 8)
and derivatised products.

| Column: | Zorbax 5μ C18(2) 150 × 4.6 mm | | | | |
|---|---|---|---|---|---|
| Mobile Phase A: | 10% acetonitrile, 0.1% formic acid | | | | |
| Mobile Phase B: | 90% acetonitrile, 0.1% formic acid | | | | |
| Flow rate: | 1 mL/min | | | | |
| Gradient: | Time 0 min | 100% | A | 0% | B |
| | Time 10 min | 0% | A | 100% | B |
| | Time 11 min | 0% | A | 100% | B |
| | Time 11.1 min | 100% | A | 0% | B |
| | Cycle time 15 min | | | | |
| Injection volume: | 20 μL | | | | |

Mass Spectrometer parameters

| Ionisation | Electrospray +ve | |
|---|---|---|
| Mass range | 250-1500 | mu |
| Capillary voltage | 3.10 | KV |
| Cone voltage | 40 | V |
| Skimmer lens offset | 5 | V |
| Ion energy | 1.4 | V |

### Example 1

### Preparation of Deoxyactagardine B (3,5-dichlorobenzylamine) monocarboxamide N-methyl glucamine salt (referred to as Example 1)

Compound 1 (500 mg) was suspended in t-butanol (250 ml) and the suspension was left to stir at 45° C. for 4 hours until all solid dissolved. A solution of N-methyl glucamine (1M aq, 492 ul) was added and the mixture was stirred for a further 1 hour. The reaction mixture was flash frozen at −80° C. and then the material was freeze dried overnight, to afford a white solid (587 mg).

### Antibacterial Activity of Example 1

Susceptibility testing with the exception of Streptococcus pneumoniae was performed by two-fold serial dilutions in Mueller Hinton Broth supplemented with 50 μg/mL Ca²⁺. Susceptibility testing of S. pneumoniae was performed by two-fold serial dilutions in Brain-Heart-Infusion Broth supplemented with 50 μg/mL Ca²⁺.

| | MIC (μg/ml) |
|---|---|
| Vancomycin-intermediate | |
| Staphylococcus aureus | |
| S. aureus V99 | 4 |
| S. aureus MI | 16 |
| S. aureus Mu3 | 8 |
| S. aureus 26 | 4 |
| S. aureus Mu50 | 4 |
| S. aureus 2 | 4 |
| S. aureus NJ | 8 |
| MRSA | |
| S. aureus R33 | 4 |
| S. aureus 12232 | 4 |
| S. aureus R36 | 4 |
| S. aureus R34 | 4 |
| S. aureus R39 | 4-8 |
| S. aureus R37 | 4 |
| S. aureus R31 | 4 |
| S. aureus R40 | 4-8 |
| S. aureus W71 | 8 |
| S. aureus W74 | 4 |
| S. aureus W82 | 4-8 |
| S. aureus W96 | 4-8 |
| S. aureus W97 | 4 |
| S. aureus W98 | 4-8 |
| S. aureus W99 | 4-8 |
| MSSA | |
| S. aureus G15 | 4 |
| S. aureus G20 | 4 |
| S. aureus G22 | 4 |
| S. aureus G23 | 4 |
| S. aureus G28 | 4 |
| S. aureus G30 | 4 |
| S. aureus G31 | 4 |
| S. aureus G32 | 4 |
| S. aureus G33 | 4 |
| S. aureus G35 | 4 |
| S. aureus G12 | 4 |
| S. aureus G26 | 4 |
| S. aureus G29 | 4 |
| S. aureus SH1000 | 4-8 |
| S. aureus 8325-4 | 4 |
| Methicillin-sensitive Staph. | |
| epidermidis | |
| S. epidermidis GRL05001 | 8 |
| S. epidermidis GRL05002 | 8 |
| S. epidermidis GRL05003 | 8 |
| S. epidermidis GRL05004 | 4-8 |
| S. epidermidis GRL05005 | 4-8 |
| S. epidermidis GRL05006 | 4-8 |
| S. epidermidis GRL05007 | <16 |
| S. epidermidis GRL05008 | 4-8 |
| S. epidermidis GRL05009 | 16 |
| S. epidermidis GRL05010 | <16 |
| S. epidermidis 9AF | 16 |
| S. epidermidis C12 | 16 |
| S. epidermidis MF87 | 4 |
| S. epidermidis C16 | 16 |
| Methicillin-resistant Staph. | |
| epidermidis | |
| S. epidermidis 7755298 | 8 |
| S. epidermidis 7865688 | 16 |
| S. epidermidis 7753921 | 8-16 |
| S. epidermidis GRL05011 | 16 |
| S. epidermidis GRL05012 | 8 |
| S. epidermidis GRL05016 | 4-8 |
| S. epidermidis GRL05017 | 16 |
| S. epidermidis GRL05013 | 8-16 |
| S. epidermidis GRL05014 | 8-16 |
| S. epidermidis GRL05015 | 8-16 |
| S. epidermidis GRL05019 | 16 |
| S. epidermidis GRL05020 | 4 |
| S. epidermidis 7864847 | <16 |
| S. epidermidis 7765349 | 4 |
| Vancomycin sensitive enterococci | |
| E. faecium 7754422 | <8 |
| E. faecium 7865229 | 16 |
| E. faecium 19579 | 4 |
| E. faecalis GRL05022 | <8 |
| E. faecalis GRL05023 | 4 |
| E. faecalis GRL05024 | <8 |
| E. faecalis GRL05026 | 8 |
| E. faecalis GRL05027 | 4-8 |
| E. faecalis GRL05029 | 4 |
| E. faecalis GRL05030 | <8 |
| E. faecalis 7757400 | <4 |
| E. faecalis 7791220 | <16 |
| Vancomycin resistant enterococci | |
| E. faecium 7662769 | <16 |
| E. faecium 7634337 | 16 |
| E. faecium 7865532 | 16 |
| E. faecium 9709024 | <16 |
| E. faecium 9710577 | 16 |
| E. faecalis GRL05031 | 2-4 |
| E. faecalis GRL05032 | 8 |
| E. faecalis GRL05033 | 8 |
| E. faecalis GRL05034 | 4 |
| E. faecalis GRL05035 | 8 |
| E. faecalis 9758512 | 8 |
| E. faecium 9704998 | 8 |
| E. faecium 7860190 | 4-8 |
| S. pyogenes | |
| S. pyogenes 7755441 | 0.06 |
| S. pyogenes 7713283 | 0.06 |
| S. pyogenes 7865253 | 0.06 |
| S. pyogenes 7757080 | <1 |
| S. pyogenes 7755255 | <2 |
| S. pyogenes 7865844 | 8 |
| S. pyogenes GRL05045 | <4 |
| S. pyogenes GRL05046 | 0.06 |
| S. pyogenes 7865289 | 0.06 |
| S. pyogenes GRL05043 | <8 |
| S. pyogenes 7755584 | 0.06-0.125 |
| S. pyogenes GRL05042 | 0.06 |
| S. pyogenes GRL05041 | <1 |
| S. pneumoniae | |
| S. pneumoniae R33 | <16 |

Modal values from up to 6 determinations

### In Vivo Efficacy of Compounds in a Mouse Bacteraemia Model.

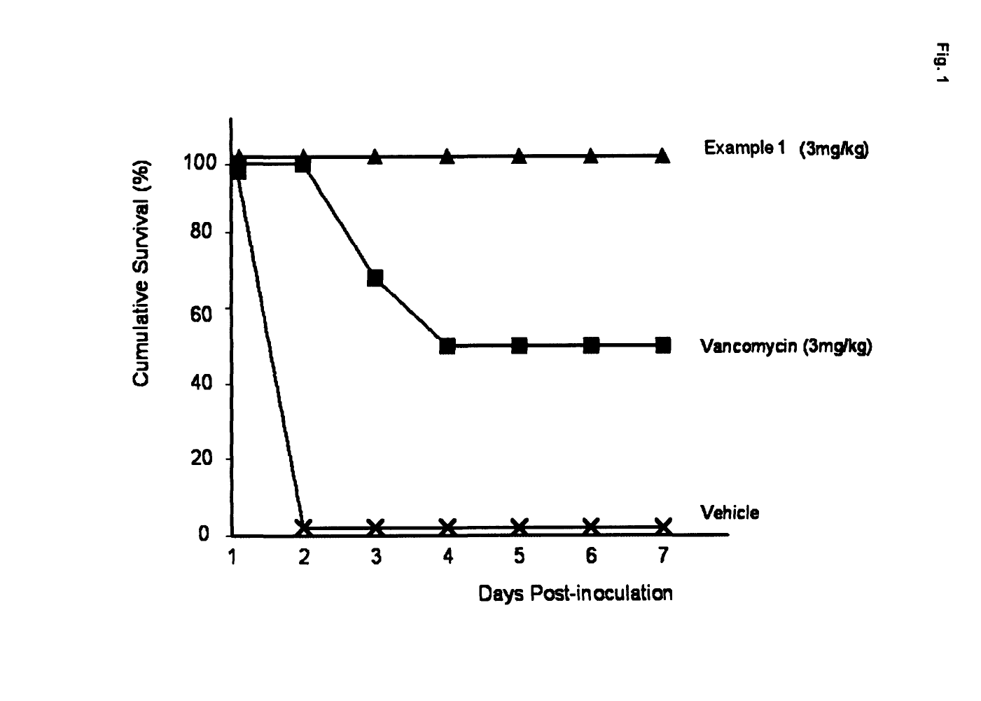

Groups of 6 male CD-1 (Crl.) derived mice weighing 24±2 g were used. Mice were inoculated intraperitoneally (IP) with an LD₉₀₋₁₀₀ of Staphylococcus aureus methicillin resistant ATCC 33591 (1.1×10⁷ CFU/mouse) in 0.5 mL of BHI broth containing 5% mucin. Example 1 and vancomycin were dissolved in 15% HPbetaCD/4.4% glucose/0.5 mM KH₂PO₄, pH 5.0 and doses of 1, 3, 5, 10 and 20 mg/Kg were administered subcutaneously (SC) to test animals at 0, 2 and 24 hour(s) after bacteria challenge. The dosing volume was 5 mL/Kg. Mortality was recorded once daily for 7 days (FIG. 1). The ED₅₀ for each compound was determined by nonlinear regression.

It was demonstrated that Example 1 at 3, 5, 10 and 20 mg/Kg×3, SC was associated with a significant antimicrobial effect against S. aureus (MRSA) in mice (at least 50% increase in survival rate) with an estimated ED₅₀ value of 1.07 mg/Kg).

Concurrently, vancomycin at 3, 5, 10 and 20 mg/Kg×3, SC exhibited significant antimicrobial effect against S. aureus (MRSA) in mice with an estimated ED₅₀ value of 3.0 mg/Kg. Mice which received Example 1 at 3 mg/Kg had a 100% survival rate.

In a second experiment Groups of 6 male CD-1 (Crl.) derived mice weighing 24±2 g were used. Mice were inoculated intraperitoneally (IP) with an LD₉₀₋₁₀₀ of Staphylococcus aureus methicillin resistant ATCC 33591 (1.35×10⁸ CFU/mouse) in 0.5 mL of BHI broth containing 5% mucin. Example 1 was dissolved in 5% dextrose/1.5 mM potassium phosphate, pH 5.0 and doses of 1, 3, 5 and 10 mg/Kg were administered intravenously (IV) to test animals at 1 and 13 hour(s) after bacteria challenge. The dosing volume was 5 mL/Kg. Mortality was recorded once daily for 7 days.

It was demonstrated that both vancomycin and Example 1 showed a dose-dependent incease in survival of mice after 7 days. For vancomycin the number of deaths at 0, 1, 3, 5 and 10 mg/kg was 5, 5, 3, 1 and 0 whereas for Example 1 the number of deaths was 5, 5, 4, 1 and 1 at these same doses.

### Efficacy of Compounds in a Neutropaenic Mouse Thigh Infection Model.

In vivo efficacy of compounds of the present invention in the treatment of bacterial tissue infections was evaluated using a neutropaenic mouse thigh model.

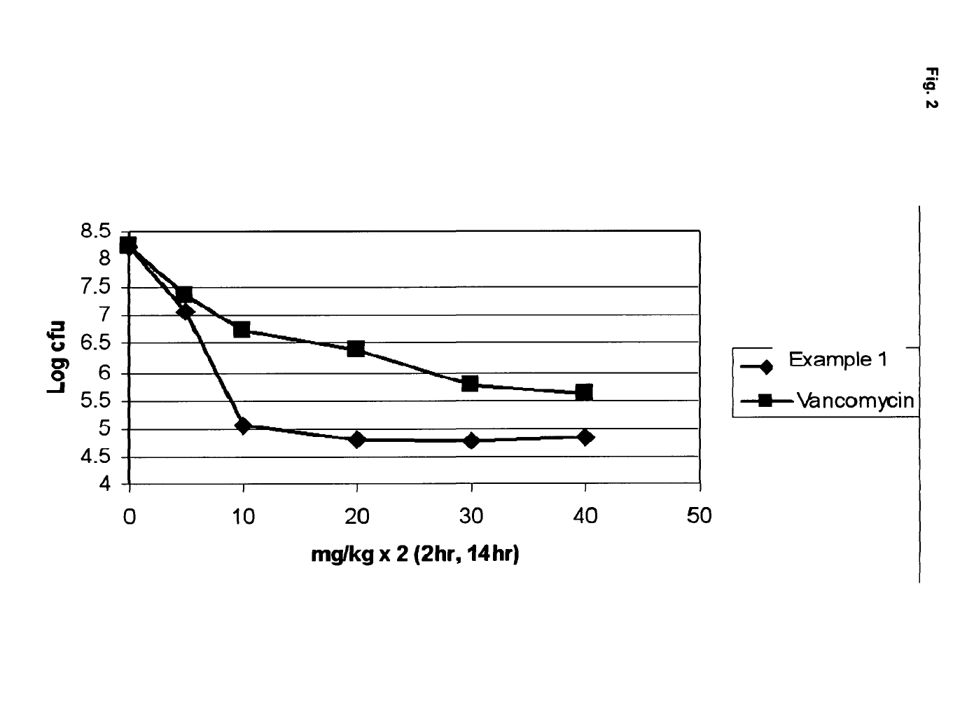

Groups of 6 male ICR mice weighing 24±2 g were used. Test animals were immunosuppressed by 2 intraperitoneal injections of cyclophosphamide, the first at 150 mg/Kg 4 days before infection (day-4) and the second at 100 mg/Kg 1 day before infection (day-1). On day 0, individual animals were inoculated intramuscularly (IM) into the right thigh of test animals with 1.15×10⁸ CFU/mouse of Methicillin Resistant Staphylococcus aureus (MRSA, ATCC 33591) suspended in 100 μL of sterile PBS, pH 7.4. Vehicle and test substances were administered intravenously (IV) at a dose volume of 6 mL/Kg, 2 and 14 hours after thigh infection. Example 1 and vancomycin were dissolved in 15% hydroxypropyl-β-cyclodextrin/4.4% glucose/1.5 mM potassium phosphate buffer, pH 7.0 and administered at doses of 5, 10, 20, 30 and 40 mg/Kg. At 26 hours after inoculation, muscle of the right thigh of each test mouse was harvested. From an additional group with no treatment, muscle of the right thigh was harvested at 2 hours after inoculation for the basal CFU determination. The removed muscle tissues were then homogenized in 3-4 mL of PBS, pH 7.4 with a ceramic mortar. Homogenates of 0.1 mL were used for serial 10-fold dilutions and plated on Mueller Hinton broth in 1.5% Bacto agar for CFU determination. It was demonstrated that Example 1 dosed IV at 5, 10, 20 30 and 40 mg/Kg×2, was associated with a significant antimicrobial effect, resulting in a >1000-fold reduction in CFU/g at 10 mg/kg and above. Concurrently, vancomycin also exhibited a significant antimicrobial effect with reductions of CFU/g of >100 fold at 30 mg/kg and above, whilst not attaining the >1000-fold reduction observed for Example 1. Results (mean cfu/g) are graphically represented in FIG. 2.

In a further experiment, groups of 6 male ICR mice weighing 24±2 g were used. Test animals were immunosuppressed by 2 intraperitoneal injections of cyclophosphamide, the first at 150 mg/Kg 4 days before infection (day-4) and the second at 100 mg/Kg 1 day before infection (day-1). On day 0, individual animals were inoculated intramuscularly (IM) into the right thigh of test animals with 1.5×10⁵ CFU/mouse of Methicillin Resistant Staphylococcus aureus (MRSA, ATCC 33591) suspended in 100 μL of sterile PBS, pH 7.4. Vehicle and test substances were administered intravenously (IV) at a dose volume of 8 mL/Kg, 2 and 14 hours after thigh infection. Example 1 was dissolved in 5% dextrose/1 mM potassium phosphate, pH 5.0 and administered at doses of 2.5, 5, 10, 15, 25 and 50 mg/Kg. At 26 hours after inoculation, muscle of the right thigh of each test mouse was harvested. From an additional group with no treatment, muscle of the right thigh was harvested at 2 hours after inoculation for the basal CFU determination. The removed muscle tissues were then homogenized in 3-4 mL of PBS, pH 7.4 with a ceramic mortar. Homogenates of 0.1 mL were used for serial 10-fold dilutions and plated on Mueller Hinton broth in 1.5% Bacto agar for CFU determination.

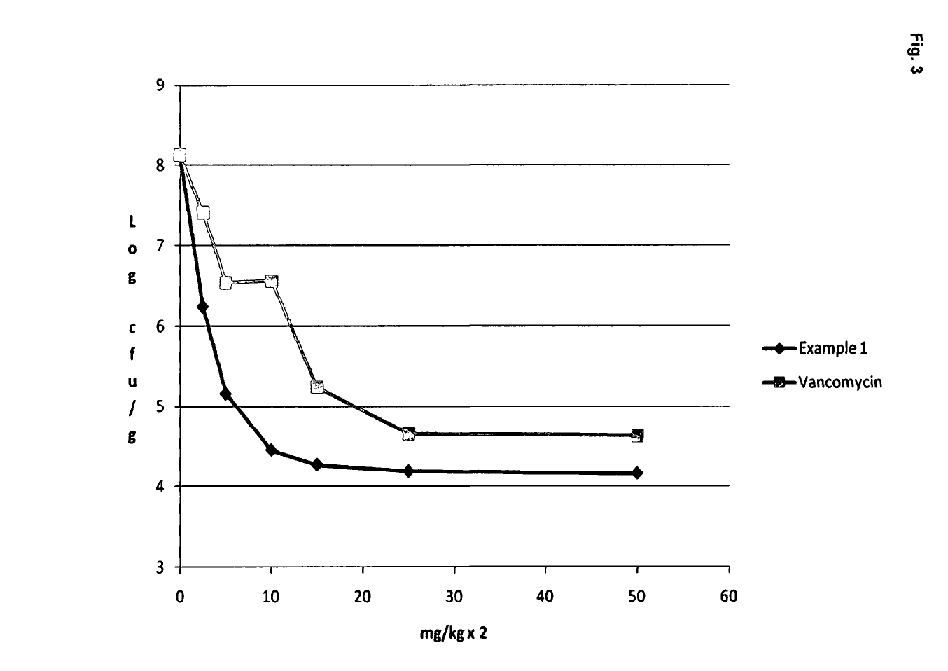

Both Example 1 and vancomycin showed a dose dependent reduction in the bacterial counts in the thigh tissue (FIG. 3).

### In Vivo Plasma Half-Life of Compounds of the Present Invention in Mice.

The in vivo half-life of Compound 1 in mice was determined by measurement of its plasma concentrations at various time points following IV dosing. 18 male CD-1 mice aged 7-9 weeks were dosed IV with a 9.3 mL/Kg dose of a 3.2 mg/mL solution of Example 1 in 15% hydroxypropyl-β-cyclodextrin/4.4% glucose/1 mM potassium phosphate (pH=7.6). Plasma samples were obtained at 10, 20, 30, 60, 120 and 240 min post-dose, sampling from 3 animals at each time point. Concentrations of Compound 1 in plasma were determined by LC-MS quantification.

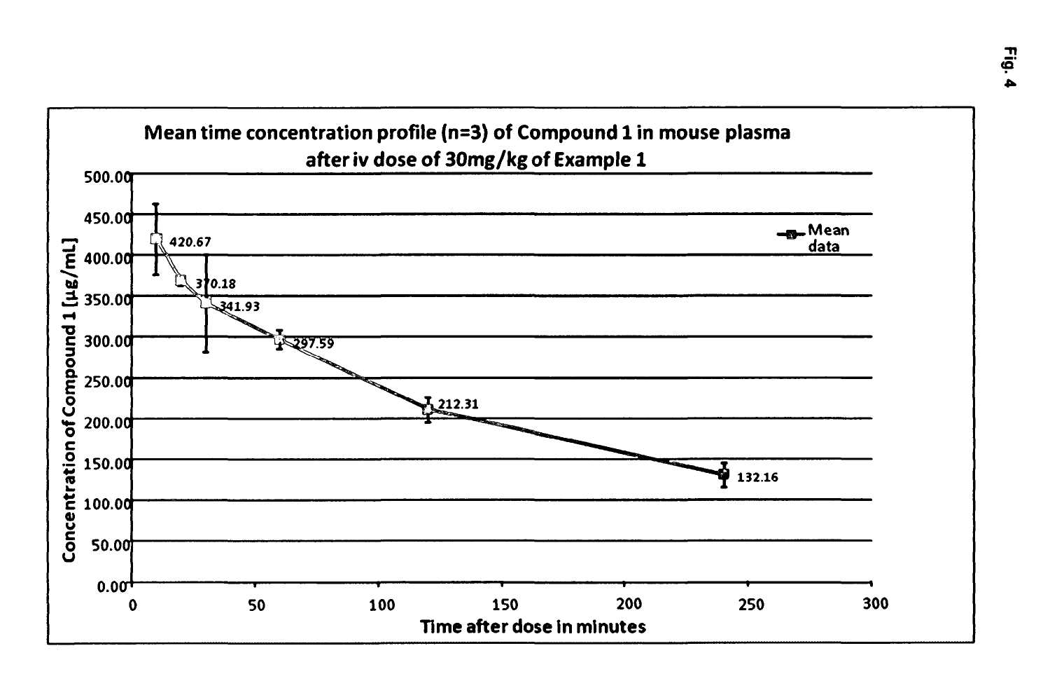

The data, summarised in FIG. 4, show that Compound 1 has a plasma half-life of approximately 2 h in the mouse.

### Solubility

Example 1 (10 mg) was dissolved in WFI (water for injection 1 or 2 mL). The solution was filtered through a Millex GP 0.2 μm filter.

The pH of 5 and 10 mg/mL solutions of Example 1 in WFI was approximately in the range pH 8.5 to 9.2.

The pH of 5 and 10 mg/mL solutions of Example 1 can be buffered into the range pH 8.0 to 8.5 using 0.5 to 1.5 mM potassium phosphate buffer pH 5.0.

The N-methyl glucamine salt of compound 1 has a solubility of more than 10 mg/mL.

The N-ethyl glucamine salt of compound 1 has a solubility of more than 10 mg/mL.

The ethanolamine salt of compound 1 has a solubility of more than 10 mg/mL.

The diethanolamine salt of compound 1 has a solubility of more than 5 mg/mL.

Below is a table of the results of a qualitative solubility assessment of solids:

TABLE 3

| | | | Total volume water added (uL) | | | | | | |
|---|---|---|---|---|---|---|---|---|---|
| Index | Solid obtained from | Nominal form | 250 | 500 | 1000 | 2000 | 4000 | 8000 | 16000 |
| 1 | NVB-333 as supplied | free acid/base | X | X | X | X | X | X | X |
| 2 | Freeze-drying from ^{t}BuOH | sodium salt | X | X | X | X | X | X | X |
| 3 | Freeze-drying from ^{t}BuOH | N-methyl-D-glucamine salt | ✓ | | | | | | |
| 4 | Freeze-drying from ^{t}BuOH | potassium salt | X | X | X | X | X | X | X |
| 5 | Freeze-drying from ^{t}BuOH | hydrochloride salt | X | X | X | X | X | X | X |
| 6 | Freeze-drying from ^{t}BuOH | mesylate salt | X | X | X | X | X | X | X |
| 7 | Freeze-drying from ^{t}BuOH | fumarate salt | X | X | X | X | X | X | X |
| 8 | Slow-evap from MeCN | sodium salt | X | X | X | X | X | X | X |
| 9 | Slow evap from MeOH | sodium salt | X | X | X | X | X | X | X |
| 10 | Slow evap from IPA | sodium salt | X | X | X | X | X | X | X |
| 11 | Slow evap from 2-methoxyethanol | sodium salt | X | X | X | X | X | X | X |
| 12 | Freeze-drying from ^{t}BuOH | lysine salt | X | X | X | X | X | X | X |
| 13 | Freeze-drying from ^{t}BuOH | arginine salt | X | X | X | X | X | X | X |
| 14 | Freezedrying from pyridine/water | sodium salt | X | X | X | X | X | X | X |
| 15 | Freezedrying from pyridine/water | potassium salt | X | X | X | X | X | X | X |

X = suspension/cloudy solution
✓ = clear solution

1. A salt of a compound of formula (I) 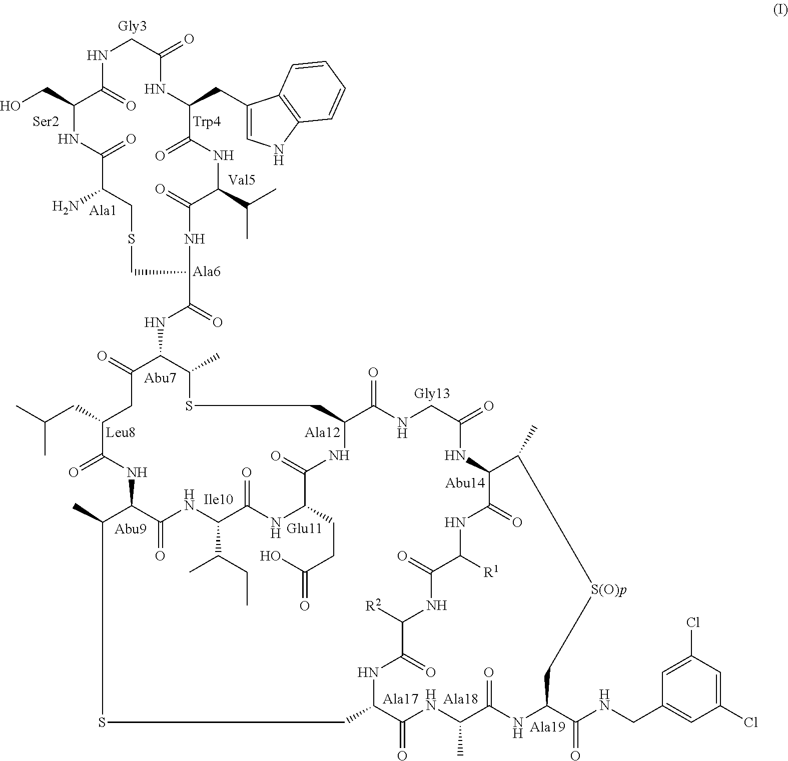
wherein
R¹ together with the carbon to which it is attached and the alpha-nitrogen and alpha carbonyl represents an amino acid residue;
R² together with the carbon to which it is attached and the alpha-nitrogen and alpha carbonyl represents an amino acid residue; and
p represents 0 or 1,
wherein the salt is formed from a small organic amine comprising at least one hydroxyl group therein.

2. A salt according to claim 1 wherein p is 1.

3. A salt according to Claim 1 wherein R¹ together with the carbon to which it is attached and the alpha-nitrogen and alpha-carbonyl represents leucine or valine.

4. A salt according to claim 1 wherein R² together with the carbon to which it is attached and the alpha-nitrogen and alpha-carbonyl represents isoleucine or valine.

5. A salt according to claim 1 wherein R¹ together with the carbon to which it is attached and the alpha-nitrogen and alpha-carbonyl represents valine and R² together with the carbon to which it is attached and the alpha-nitrogen and alpha-carbonyl represents isoleucine.

6. A salt according to claim 1 wherein R¹ together with the carbon to which it is attached and the alpha-nitrogen and alpha-carbonyl represents leucine and R² together with the carbon to which it is attached and the alpha-nitrogen and alpha-carbonyl represents valine.

7. A salt according to claim 1 wherein said compound has the formula:

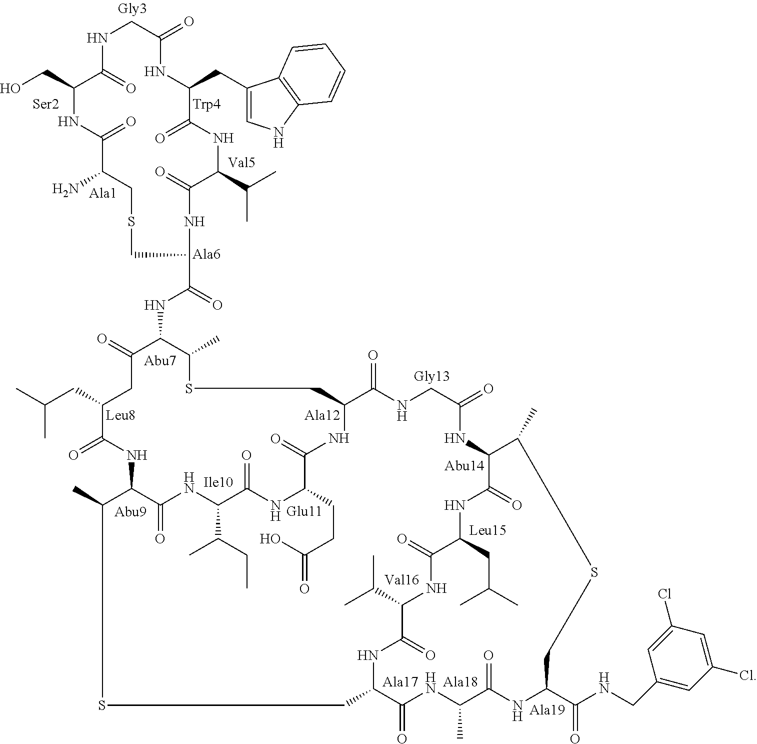

8. A salt according to claim 1 wherein the compound from which the salt is formed is an organic amine.

9. A salt according to claim 1 wherein the compound comprises 10% w/w water or less.

10. A salt according to claim wherein the salt comprises 5% w/w or less of t-butanol.

11. A salt according to claim 8 wherein the organic amine compound from which the salt is formed comprises no more than 6 carbon atoms.

12. A salt according to claim 8 wherein the organic amine comprises one, two, three, four or five hydroxyl groups.

13. A salt according to claim 12 wherein a hydroxyl in the organic amine is bonded to a carbonyl therein to form an acid, at least in the starting material from which the salt is formed.

14. A salt according to claim 12 wherein the organic amine is selected from the group consisting of glucamine, ethanolamine, diethanolamine and arginine.

15. A salt according to claim 14 wherein the organic amine is a glucamine.

16. A salt according to claim 15 wherein the glucamine is N-methyl glucamine.

17. A salt according to claim 15 wherein the glucamine is N-ethyl glucamine.

18. A salt according to claim 14 wherein the organic amine is ethanolamine

19. A salt according to claim 14 wherein the organic amine is arginine.

20. A salt according to claim 1, wherein the salt is a solvate.

21. A process for producing a salt according to claim 1 comprising:
a) dissolving the parent compound in a suitable solvent in the presence of an appropriate amount of salt-forming partner; and
b) lyophilising the product of step a); or
c) evaporating the solvent

22. A process according to claim 21 wherein said solvent is t-butanol and/or DMSO.

23. A process according to claim 21 wherein the solvent is t-butanol and water.

24. A process according to claim 21 wherein the salt-forming partner is provided as a 1 to 2 equivalent.

25. A process wherein the solution of step a) in claim 21 is spray dried with one or more excipients to provide particles that are agglomerations of the salt and the excipients.

26. A pharmaceutical composition comprising a therapeutically effective amount of a salt according to claim 1 and a pharmaceutically acceptable excipient, diluent and/or carrier.

27. (canceled)

28. A process for preparing a pharmaceutical composition according to claim 26 comprising mixing the salt, together with a pharmaceutically acceptable excipient, diluent and/or carrier.

29. A salt according to claim 1 in combination with a further therapeutic agent.

30. A pharmaceutical composition according to claim 26 in combination with a further therapeutic agent.

31. (canceled)

32. A method of treatment or prophylaxis of a microbial infection comprising administering a therapeutically effective amount of a salt according to claim 1 to a patient in need thereof.

33. The method of claim 32 wherein the microbial infection is a skin infection.

34. The method of claim 32 wherein the microbial infection is a gram positive microbial infection.

35. The method of claim 34 wherein the gram positive microbial infection is selected from the group consisting of S. aureus, E. faecalis, E. faecium, S. pyogenes, S. pneumoniae and C. difficile.

36. The method of claim 35 wherein the gram positive microbial infection is Staphylococcus aureus.

37. The method of claim 34 wherein the gram positive microbial infection is methicillin resistant Staphylococcus aureus.


SEQUENCE LISTING (8 sequences)

SEQ ID NO: 1 - protein, 19 aa, Artificial sequence
  Synthetic sequence
ASGWVATLTIEAGTXXAAA

SEQ ID NO: 2 - protein, 19 aa, Artificial sequence
  Synthetic sequence
ASGWVATLTIEAGTLVAAA

SEQ ID NO: 3 - protein, 19 aa, Artificial sequence
  Synthetic sequence
ASGWVATLTIEAGTLXAAA

SEQ ID NO: 4 - protein, 19 aa, Artificial sequence
  Synthetic sequence
ASGWVATLTIEAGTVXAAA

SEQ ID NO: 5 - protein, 19 aa, Artificial sequence
  Synthetic sequence
ASGWVATLTIEAGTXIAAA

SEQ ID NO: 6 - protein, 19 aa, Artificial sequence
  Synthetic sequence
ASGWVATLTIEAGTXVAAA

SEQ ID NO: 7 - protein, 19 aa, Artificial sequence
  Synthetic sequence
ASGWVATLTIEAGTVIAAA

SEQ ID NO: 8 - protein, 19 aa, Artificial sequence
  Synthetic sequence
ASGWVATLTIEAGTLVAAA